(19) United States
(12) Patent Application Publication
(10) Pub. No.: US 2025/0137996 A1
(43) Pub. Date: May 1, 2025

(54) HIGH-THROUGHPUT IN VITRO LUNG INJURY MODEL SYSTEM FOR SCREENING A PLURALITY OF CANDIDATE COMPOUNDS FOR THE TREATMENT OF RESPIRATORY INFECTIOUS DISEASES

(71) Applicant: Centre for Immunology & Infection Limited, Hong Kong (HK)

(72) Inventors: Chi Wai Michael CHAN, Hong Kong (HK); Po Yee MA, Hong Kong (HK); Hiu Ha CHING, Hong Kong (HK)

(21) Appl. No.: 18/919,477

(22) Filed: Oct. 18, 2024

Related U.S. Application Data
(60) Provisional application No. 63/593,242, filed on Oct. 26, 2023.

(51) Int. Cl. G01N 33/50; C12M 1/32; C12N 5/071

(52) U.S. Cl. CPC G01N 33/5044; C12M 23/12; C12N 5/0688; C12N 2503/02

(57) ABSTRACT

The present invention relates to a physiologically relevant, high-throughput in vitro human lung injury model used for studying therapeutic interventions against severe respiratory pathogens. In this model, human alveolar epithelial cells are infected with the highly pathogenic influenza A virus A/HK/483/97 (H5N1) and subsequently treated with extracellular vesicles (EVs) 24 hours post-infection. Influenza A(H5N1) infection significantly reduces alveolar fluid clearance (AFC) and increases alveolar permeability (APP) after 24 hours. The administration of therapeutic EVs restores both AFC and APP in vitro, reduces the expression of dysregulated proinflammatory cytokines, and enhances virus-suppressed alveolar sodium and chloride transporters in the infected epithelial cells. These findings demonstrate the potential therapeutic efficacy of EVs in alleviating epithelial damage caused by influenza A(H5N1) and underscore the applicability of the high-throughput lung model for screening treatments for respiratory diseases.

### CROSS-REFERENCE TO RELATED APPLICATIONS

The present application claims the priorities from the U.S. provisional patent application Ser. No. 63/593,242 filed Oct. 26, 2023, and the disclosure of which is incorporated herein by reference in its entirety.

### FIELD OF THE INVENTION

The present invention pertains to the field of in vitro disease models and high-throughput screening methodologies.

### BACKGROUND OF THE INVENTION

The airway epithelium in human lungs serves as both a physical and immunological barrier against exterior pathogens, protecting underlying tissues from infection. In many respiratory diseases, the integrity of the airway epithelium is compromised, the process of which is difficult to study in the human lungs outside of the clinical setting. Influenza virus infections of human lungs often result in chronic damage of the alveolar epithelium, which maintains proper alveolus function such as gas exchange. Tight junctions between alveolar cells form a robust epithelial layer capable of regeneration after damage.

While animal models have been widely used to study acute lung injury, they present challenges due to interspecies differences in pulmonary anatomy and immune response, which limit the translational value of the findings. Ethical issues also continue to plague the use of live animals in pharmaceutical research. Consequently, a reliable and physiologically relevant in vitro model of the human alveolar epithelium is instrumental/platform for rapid drug discovery, to support pre-clinical research addressing causes and treatment of respiratory diseases.

In order to understand the mechanisms driving disease initiation and progression, it is important to have a predictive in vitro modelling system resembling the human physiological condition outside of the clinical setting. Initial in vitro models using submerged cultures of human lung epithelial cells have been limited in their ability to replicate human lung physiology. The introduction of transwell systems, which allow air-liquid or liquid-liquid interfaces, better mimic the human respiratory tract environment. However, the use of immortalized cell lines, such as A549 cells derived from lung carcinoma, fails to reproduce key physiological features due to phenotypical differences in culture conditions. Moreover, the availability of non-diseased human primary alveolar cells in sufficient quantities is limited and costly.

Given these limitations, there is a need for a scalable and reproducible in vitro model that can mimic human lung physiology and provide an efficient platform for screening potential therapeutic interventions. The current invention addresses this need.

### SUMMARY OF THE INVENTION

The present invention aims to offer a high-throughput in vitro lung injury model, optimized for culturing primary human alveolar epithelial cells. This model facilitates the study of mechanisms involved in highly pathogenic influenza virus infections and enhances drug screening capabilities, while reducing the demand for donor cells and minimizing the time required.

In accordance with one aspect of the present invention, the present invention provides a high-throughput in vitro lung injury model system. Such a system provides a more predictive and scalable in vitro model of the human alveolar epithelium, facilitating the study of disease mechanisms and the development of therapeutic interventions. The system is used for screening a plurality of candidate compounds for the treatment of respiratory infectious diseases. The system includes a plurality of transwell inserts in a 96-well system, a device for applying a plurality of candidate compounds to infected alveolar epithelial cells, and an apparatus for measuring alveolar fluid clearance and alveolar protein permeability in the infected alveolar epithelial cells. Each transwell insert contains a monolayer of primary human alveolar epithelial cells, and the primary human alveolar epithelial cells are infected with one or more respiratory pathogens. The tight junction integrity of the alveolar epithelial cells is monitored by measuring transepithelial electrical resistance ( TEER). The TEER is maintained at a value of at least 2000 Ω/cm².

The AFC is measured as a function of the concentration of fluorescence-labeled dextran present in the apical chamber after a 24-hour incubation period. The APP is measured using fluorescence-labeled dextran with a molecular weight of 70 kDa. The fluorescence-labeled dextran is used to track the unidirectional flux from the apical to the basolateral chamber of the transwell.

In accordance with one aspect of the present invention, the respiratory infectious diseases include viral respiratory infections.

In accordance with one aspect of the present invention, the one or more respiratory pathogens include influenza virus, respiratory syncytial virus (RSV), coronavirus, middle east respiratory syndrome coronavirus (MERS-COV), human parainfluenza viruses (HPIVs), human metapneumovirus (hMPV), adenoviruses, rhinoviruses, and enteroviruses.

In accordance with one aspect of the present invention, the primary human alveolar epithelial cells are isolated from a non-malignant lung tissue.

In accordance with one aspect of the present invention, the primary human alveolar epithelial cells are seeded in the transwell inserts at a density of 1×10⁴ to 1×10⁵ cells per well.

In accordance with one aspect of the present invention, the plurality of candidate compounds include nano-sized extracellular vesicles (EVs) derived from human mesenchymal stromal cells.

In accordance with one aspect of the present invention, the present invention provides a method for high-throughput screening of a plurality of candidate compounds for the treatment of respiratory infectious diseases, including: isolating primary human alveolar epithelial cells from a non-malignant lung tissue; seeding and culturing the primary human alveolar epithelial cells in a plurality of transwell inserts in a 96-well transwell system; infecting the primary human alveolar epithelial cells with one or more respiratory pathogens; treating infected primary human alveolar epithelial cells with a plurality of candidate compounds; and assessing effect of the plurality of candidate compounds on at least one physiological parameter comprising alveolar fluid clearance (AFC) and alveolar protein permeability (APP) to screening candidate compounds.

In accordance with one aspect of the present invention, the respiratory infectious diseases include viral respiratory infections.

In accordance with one aspect of the present invention, the one or more respiratory pathogens include influenza virus, respiratory syncytial virus (RSV), coronavirus, middle east respiratory syndrome coronavirus (MERS-COV), human parainfluenza viruses (HPIVs), human metapneumovirus (hMPV), adenoviruses, rhinoviruses, and enteroviruses.

In accordance with one aspect of the present invention, the primary human alveolar epithelial cells are isolated from a non-malignant lung tissue.

In accordance with one aspect of the present invention, the plurality of candidate compounds include nano-sized EVs derived from human mesenchymal stromal cells.

In accordance with one aspect of the present invention, the step of measuring impact of the therapeutic compound on at least one physiological parameter includes measuring inflammatory cytokine expression to evaluate the therapeutic effect of the candidate compound.

In accordance with one aspect of the present invention, the step of measuring impact of the therapeutic compound on at least one physiological parameter includes monitoring activity of one or more ion transporters selected from the group consisting of epithelial sodium channels (ENaC), CFTR channels, and Na/K-ATPase pumps.

In accordance with one aspect of the present invention, the primary human alveolar epithelial cells are cultured in a liquid-liquid interface within the 96-well transwell system.

In accordance with one aspect of the present invention, the primary human alveolar epithelial cells are seeded at a density of 1×10⁴ to 1×10⁵ cells per well.

In accordance with one aspect of the present invention, the primary human alveolar epithelial cells are infected with the one or more respiratory pathogens at a multiplicity of infection (MOI) ranging from 0.1 to 3.

In accordance with one aspect of the present invention, the plurality of candidate compounds are administered at a concentration ranging from 1×10⁷ to 1×10⁹ EV particles for each well.

In accordance with another aspect of the present invention, the method includes creation of an in vitro alveolar epithelium displaying tight junction formation in a monolayer to set up the in vitro lung injury model in a high throughput format (96 wells).

In accordance with another aspect of the present invention, the method includes investigation of the mechanisms involved in influenza virus infection and screening effective drugs for treatment.

This invention utilizes primary culture of cells isolated from human lung tissues seeded into a 96-wells format of a transwell system, which increases the physiological relevance of the in vitro acute lung injury model and allows for rapid and high throughput screening and identification of patient-specific drug treatments.

This high-throughput in vitro lung injury model is good for patient-specific therapy as the primary human lung cells are acquired from a specific donor. This allows tailored treatment of respiratory diseases based on the fine-tuning of phenotypic drug screening in the in vitro lung injury model, which is readily translatable to the clinical setting, therefore maximizing the chance of successful treatment.

Furthermore, the high throughput format (96-wells) transwell system allows screening of larger scale of drug/intervention candidate at the same time. This 96-wells format allows us to utilize the minimum amount of primary human lung cells in each individual transwell, which maximizes the screening capability.

### BRIEF DESCRIPTION OF THE DRAWINGS

Embodiments of the invention are described in more details hereinafter with reference to the drawings, in which:

FIG. 1A shows a schematics of the in vitro acute lung injury model using confluent monolayer of human alveolar epithelial cells (AEC) seeded into the apical chamber of each transwell to mimic the human alveolus in clinical setting;

FIG. 1B shows the daily monitoring of tight junction integrity in cells, achieved by measuring transepithelial electrical resistance (TEER) using an EVOM machine;

FIG. 1C depicts TEER values of AEC observed across a six-day period before experiment;

FIG. 1D shows alveolar fluid clearance (AFC) and an increase in alveolar protein permeability (APP) upon infection with highly pathogenic influenza virus A(H5N1). Cytomix containing proinflammatory cytokines (IL-1β, TNF-α, and IFN-γ) is used as an injury-inducing positive control. Data are the mean±SD from three experiments. *p≤0.05; **p≤0.01; ***p≤0.001 and ****p≤0.0001;

FIG. 2A shows the AFC of infected cells with or without EV treatment, compared to untreated infected cells;

FIG. 2B shows the APP of infected cells with or without EV treatment, compared to untreated infected cells;

FIG. 2C shows expression of different inflammatory cytokines of A(H5N1)-infected cells with or without EV treatment. Data represent the mean±SD from three experiments. *p≤0.05; **p≤0.01 and ***p≤0.001;

FIG. 2D shows activity of different ion transporters of A(H5N1)-infected cells with or without EV treatment. Data represent the mean±SD from three experiments. *p≤0.05; **p≤0.01 and ***p≤0.001.

### DETAILED DESCRIPTION

It is difficult to examine the physiological changes and related mechanisms of emerging respiratory virus-induced acute lung injury outside of clinical settings. Therefore, it is important to accurately portray this in an in vitro setting with physiological relevance, in order to advance drug screening for precision and personalized medicine.

Accordingly, the present invention provides a high-throughput in vitro lung injury model that replicates the physiological conditions of the human alveolar epithelium, using a 96-well transwell system seeded with primary human alveolar epithelial cells (AECs). This model facilitates rapid screening of therapeutic compounds while minimizing the use of primary cells. The invention allows for the study of respiratory pathogens, such as highly pathogenic influenza viruses, and the screening of potential therapeutic compounds. This system enables researchers to measure key physiological parameters, including AFC and APP, both of which are impacted during acute lung injury.

One aspect of the invention involves the isolation and culture of primary human alveolar epithelial cells from a non-malignant lung tissue, which are then seeded into a transwell system where they form a monolayer with tight junction integrity to mimic the human alveolus. Upon infection with a respiratory pathogen, the monolayer exhibits decreased AFC and increased APP, thereby mimicking the conditions observed in acute lung injury. AFC is assessed by tracking fluorescence-labeled dextran movement across the epithelial layer, while APP is evaluated based on the unidirectional flux of dextran from the apical to the basolateral chamber. This allows for real-time monitoring of the lung epithelium's response to infection and treatment.

In one embodiment, the model monitors TEER to assess tight junction integrity, ensuring values remain above 2000 Ω/cm², an indicator of proper barrier function.

In one embodiment, the AECs are isolated from a non-malignant lung tissue.

In one embodiment, the seeding density of AEC cells is in a range of 1×10⁴ to 1×10⁵ cells.

Preferably, the seeding density of AEC cells is 5×10⁴ cells.

The primary human AECs are infected with one or more respiratory pathogens at a multiplicity of infection (MOI) ranging from 0.1 to 3. The respiratory pathogens may include influenza A(H5N1) virus, respiratory syncytial virus (RSV), coronavirus, middle east respiratory syndrome coronavirus (MERS-COV), human parainfluenza viruses (HPIVs), human metapneumovirus (hMPV), adenoviruses, rhinoviruses, and enteroviruses. These respiratory pathogens are used to infect the alveolar epithelial cells.

Preferably, the respiratory pathogen is highly pathogenic influenza A virus A/HK/483/97 (H5N1).

In one embodiment, nano-sized EVs are used as therapeutic compounds. These EVs have been shown to reverse the harmful effects of pathogens like influenza A(H5N1) by restoring AFC and reducing APP. The EV treatment modulates the inflammatory response and improves ion transporter function, such as epithelial sodium channels and CFTR channels, which are typically downregulated during infection. Apart from EVs, other potential therapeutic drugs including but not limited to therapeutic proteins and medicinal compounds and their derivatives can be evaluated and used.

The present invention also provides a method for high-throughput screening of a plurality of candidate compounds for the treatment of respiratory infectious diseases. The method includes isolating primary human alveolar epithelial cells from a non-malignant lung tissue; seeding and culturing the primary human alveolar epithelial cells in a plurality of transwell inserts in a 96-well transwell system; infecting the primary human alveolar epithelial cells with one or more respiratory pathogens; treating infected primary human alveolar epithelial cells with a plurality of candidate compounds; and assessing effect of the plurality of candidate compounds on at least one physiological parameter comprising alveolar fluid clearance (AFC) and alveolar protein permeability (APP) to screening candidate compounds.

In one embodiment, the step of measuring impact of the therapeutic compound on at least one physiological parameter including measuring inflammatory cytokine expression. The inflammatory cytokine may include IFNβ, IL-1β, RANTES, IP-10, MCP-1, IL-6, IL-8, and TNFα.

In one embodiment, the step of measuring impact of the therapeutic compound on at least one physiological parameter including monitoring activity of one or more ion transporters. The ion transporters may include CFTR, Alpha ENaC, Beta ENaC, Gamma ENaC, Alpha1 NaKATPase, Beta1 NaKATPase, and Beta3 NaKATPase.

The present invention provides several advantages over conventional lung injury models, including scalability, reproducibility, and the potential for personalized therapy.

The 96-well transwell format allows for the simultaneous screening of multiple candidate compounds, increasing the efficiency of the drug discovery process.

The invention reduces the need for large amounts of primary human cells by optimizing cell seeding densities, making it more cost-effective and accessible. By using primary cells derived from specific donors, this model facilitates patient-specific drug screening, increasing the likelihood of successful clinical translation. Its high-throughput design maximizes the use of limited primary cell resources, enabling the screening of a large number of therapeutic compounds in parallel. This approach is particularly useful for identifying effective treatments for respiratory diseases caused by emerging pathogens.

### EXAMPLE

### Example 1

### Materials and Method

### Virus

Influenza virus A/HK/483/97 (H5N1) is used for infection. All influenza viruses are passaged in Madin-Darby Canine Kidney (MDCK) cells. Viral titers are determined by median tissue culture infectious dose (TCID₅₀). All experiments are performed inside a biosafety level 3 facility.

### Isolation of Human Alveolar Epithelial Cells

Human alveolar epithelial cells are isolated from resected, non-malignant human lung tissue. Briefly, the visible bronchi are removed, and the lung tissue is minced into pieces of less than 0.5 mm in size. Minced tissue is washed three times with Hank's balanced salt solution (HBSS) (Gibco) and 0.7 mM sodium bicarbonate (Gibco) at pH 7.4 to remove most of the macrophages and blood cells. The tissue is digested using a combination of 0.5% trypsin (Gibco) and 4 U/mL elastase (Worthington Biochemical Corp.) for 40 minutes at 37° C. in a shaking water bath. Digestion is stopped by adding DMEM/F12 medium (Gibco) with 40% FBS and 350 U/mL DNase I (Sigma). Cell clumps are dispersed by repeatedly pipetting the cell suspension for 10 minutes. A disposable cell strainer (gauze size 50 μm) (BD Bioscience) is used to remove large undigested tissue fragments. The single-cell suspension is centrifuged, and the pellet is resuspended in a 1:1 mixture of DMEM/F12 medium and small airway basal medium (Lonza) supplemented with 0.5 ng/ml epidermal growth factor, 500 ng/mL epinephrine, 10 μg/mL transferrin, 5 μg/mL insulin, 0.1 ng/mL retinoic acid, 6.5 ng/mL triiodothyronine, 0.5 μg/mL hydrocortisone, 30 μg/mL bovine pituitary extract, 0.5 mg/mL BSA, 5% FBS and 350 U/mL DNase I. The cell suspension is plated in tissue culture flasks and incubated in a 37° C. water-jacketed incubator at 5% CO₂ for 90 minutes. The non-adherent cells are layered on a discontinuous cold Percoll density gradient (density 1.089 and 1.040 g/mL) and centrifuged at 25×g for 20 minutes. The cell layer at the interface of the two gradients is collected and washed four times with HBSS to remove the Percoll fluid. The cell suspension is incubated with magnetic beads coated with anti-CD14 antibodies (MACS CD14 MicroBeads) at room temperature for 20 minutes. After removal of the beads by using a magnet, cell viability is assessed by trypan blue dye exclusion. The alveolar epithelial cell suspension is resuspended in SAGM (Lonza) supplemented with 1% FBS, 100 U/mL penicillin and 100 μg/mL streptomycin and plated at a cell density of 3×10⁵ per cm². The cells are maintained in a humidified atmosphere (5% CO₂, 37° C.), and growth medium is changed daily, starting 60 hours after plating the cells. When the cell layer approached 75% confluence, the alveolar epithelial cells are dissociated with trypsin, detached into HBSS, and then seeded as described below in the transwell culture inserts.

### Example 2

### In Vitro Model Setup

To study the effect of one or more respiratory pathogens on alveolar fluid clearance and protein permeability in human alveolar epithelial cells, this example adapts a physiologically relevant 24-transwell in vitro acute lung injury model into the high-throughput 96-transwell format for use in a BSL-3 laboratory.

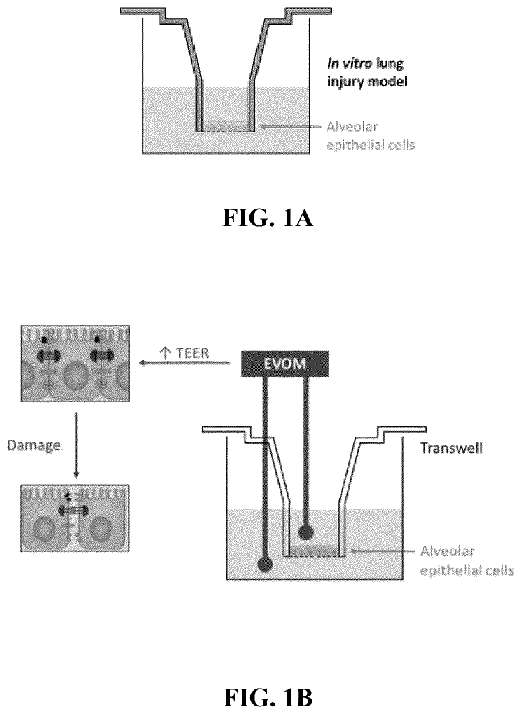

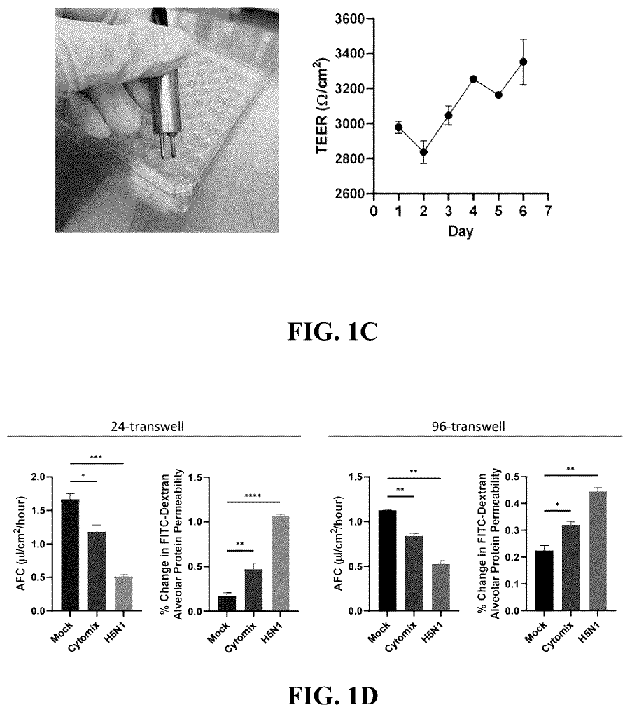

FIG. 1A depicts the establishment of the high-throughput in vitro lung injury model. Primary human alveolar epithelial cells are isolated from resected, non-malignant lung tissue. The isolation process involves enzymatic digestion of the lung tissue using trypsin and elastase, followed by filtration and density gradient centrifugation to obtain a purified population of alveolar epithelial cells. After that, the alveolar epithelial cells are plated on the apical chamber of 96-well Costar Transwell inserts with a 0.4-μm pore size (Corning), at a density of 5×10⁴ cells per well. The microporous transwell membrane establishes a liquid-liquid interface similar to that of human lung epithelium, and the plated cells are maintained in a humidified atmosphere (5% CO₂, 37° C.). Transepithelial resistance is maintained at ≥2,000 Ω/cm², which indicates sufficient tight junction integrity between cells, ensuring that the monolayer closely mimics the alveolar barrier found in human lungs. In addition, cytomix is used at 50 ng/ml as a positive control for lung injury, which is a mixture of the cytokines IL-1β, TNF-α, and IFN-γ (R&D Systems).

The tight junction integrity between AECs is monitored daily with an epithelial voltohmmeter (EVOM). The transepithelial resistance increases with the formation of tight junctions, which decreases upon monolayer damage (FIG. 1B). TEER is observed to increase throughout a 6-day period before experiments are performed on the established in vitro lung model (FIG. 1C).

Acute lung injury arising from influenza A(H5N1) virus infection is responsible for a high mortality rate, for which few current therapeutic options are available. Key characteristics of acute lung injury include impaired AFC and increased APP observed in the damaged lung alveolar epithelium.

Net alveolar fluid transport from the apical chamber of the transwell culture insert (containing a monolayer of alveolar epithelial cells infected with influenza virus) to the basolateral chamber of the transwell at 24 hours post infection is measured. In this model, the AECs are inoculated with the respective influenza A viruses at a MOI of 0.1 for 1 hour. Then, 130 μl of FITC-labeled dextran (Sigma) with a size of 70 kDa is added to the cells (final concentration, 500 ng/μl). After 5 minutes, a sample of 100 μl is collected from the apical chamber for initial FITC measurement, after which it is transferred back into the apical chamber for overnight incubation. After 24 hours of dextran incubation, 100 μl is collected from the apical chamber, while 100 μl is collected from the basolateral chamber for the final FITC measurement. The fluorescence of each sample is measured by a modulus fluorometer (FLUOstar OPTIMA, BMG Labtech) at excitation wavelength of 485 nm and emission wavelength of 520 nm. A standard curve of known FITC concentration is constructed for the calculation of FITC-dextran present in the transwell chambers at different time points. The net alveolar fluid transport across the epithelial monolayer is measured as [1−(FITC concentration in the initial apical sample/FITC concentration in the final apical sample)]/130 μl/0.143 cm²/24 hours. The final basolateral reading is collected after 24 hours of dextran incubation to measure protein permeability, based on the unidirectional flux of fluorescence-labeled dextran from the apical to the basolateral chamber of the transwell culture insert.

Referring to FIG. 1D, infection of the AECs results in a significant reduction in AFC and an increase in APP at 24 hours post-infection compared to the mock group. These effects are assessed using fluorescence-labeled dextran to monitor fluid transport and protein leakage across the monolayer. Cytomix serves as a positive control for monolayer damage.

Candidate therapeutic compounds, such as 500 ng/μl nano-sized extracellular vesicles (EVs), are introduced to the infected cells to evaluate their therapeutic potential. The EVs, derived from human mesenchymal stromal cells, are added to the transwell system post-infection, and their effects on AFC and APP are measured after a 24-hour incubation period. A baseline fluorescence reading is taken, and the dextran is allowed to incubate with the monolayer for 24 hours. The final fluorescence readings from both the apical and basolateral chambers are used to calculate the net alveolar fluid transport across the monolayer. Alveolar protein permeability is measured by tracking the unidirectional flux of the fluorescence-labeled dextran from the apical to the basolateral chamber.

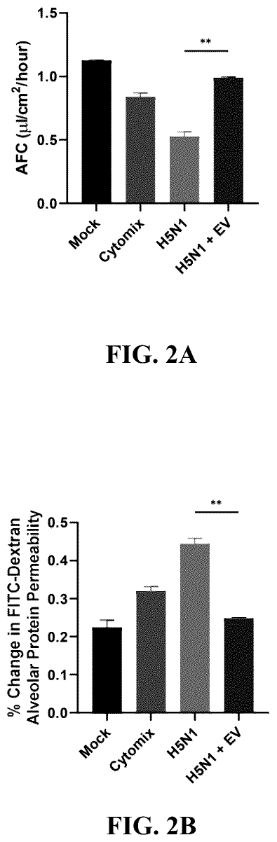

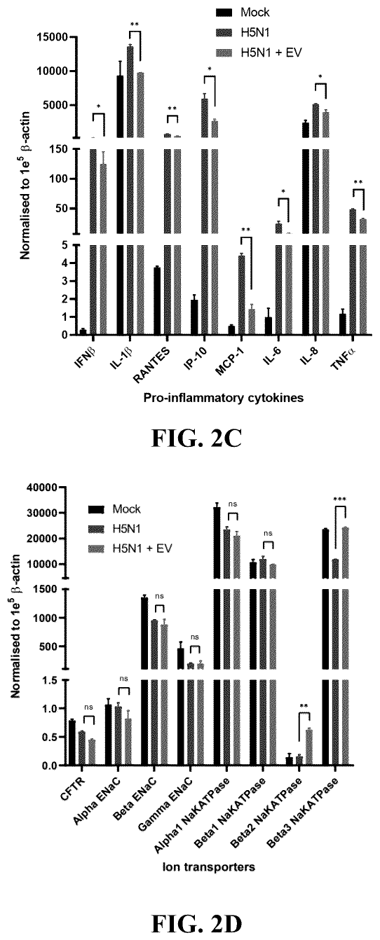

The administration of EVs is found to restore AFC and reduce APP in the infected cells, indicating their therapeutic efficacy in treating lung injury caused by respiratory pathogens. Referring to FIGS. 2A-2B, after EV treatment, AFC is significantly restored and APP is reduced compared to untreated infected cells.

Moreover, the AECs in transwells are infected with A(H5N1) influenza virus at MOI of 2 to investigate the gene expressions of cytokines and ion transporters upon virus infection and potential therapeutic treatment. The results show that EV treatment reverses the expression of pro-inflammatory cytokines in the A(H5N1)-infected cells, as shown in FIG. 2C, where pro-inflammatory cytokine levels such as IL-1β, TNF-α, and IFN-β are reduced.

The activity of key ion transporters, including the epithelial sodium channel (ENaC), CFTR channels, and Na/K-ATPase pumps, is also monitored to determine their role in fluid clearance and epithelial barrier function. Turning to FIG. 2D, the results show that pathogen-induced downregulation of these ion channels and pumps is a hallmark of lung injury, which can be reversed upon therapeutic intervention with EVs.

Influenza virus infection has been found to disrupt alveolar fluid transport by inhibiting epithelial sodium channel activity through interaction of viral hemagglutinin and matrix proteins with the epithelial sodium channel. The activation of proinflammatory pathways is also shown to be responsible for the downregulation of ion transporters responsible for vectorial fluid transport across the alveolar epithelium. Indeed, the present data also indicates that upon A(H5N1) infection, the increased level of pro-inflammatory cytokines correlate to the decreased expressions of CFTR, sodium ENAC channels and sodium and potassium Na/K-ATPase pumps, which suggests that the impaired AFC related to the impaired activity of the ion transporters in the alveolar epithelium.

In summary, the present invention establishes a physiologically relevant high-throughput in vitro lung injury model to maximize the efficiency of therapeutic molecule screening for severe human influenza. By using this model, it demonstrated that highly pathogenic A(H5N1) virus significantly impaired AFC and APP in cultured human alveolar epithelial cells by promoting pro-inflammatory cytokine production and downregulating alveolar ion transporter activity. This pathology is subdued by the addition of EVs in vitro, indicating their therapeutic potential for treating diseases arising from A(H5N1) infection.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments are chosen and described in order to best explain the principles of the invention and its practical application, thereby enabling others skilled in the art to understand the invention for various embodiments and with various modifications that are suited to the particular use contemplated.

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

“Transepithelial Electrical Resistance (TEER)” is a quantitative measurement of the electrical resistance across a monolayer of cells, typically used to assess the integrity and permeability of tight junctions in epithelial or endothelial cell cultures. TEER provides a direct indicator of the strength of the barrier function formed by the cells. Higher TEER values indicate stronger tight junctions and lower permeability, reflecting a more intact and functional epithelial or endothelial barrier, while lower TEER values suggest weakened junctions and increased permeability. In in vitro models, TEER is commonly used to monitor the health and integrity of cell monolayers over time.

“Respiratory infectious diseases” are illnesses caused by pathogens that infect the respiratory system, which includes the nose, throat, sinuses, airways, and lungs. These diseases are typically spread through droplets from coughing, sneezing, talking, or direct contact with contaminated surfaces. They can range in severity from mild colds to life-threatening conditions, depending on the pathogen and the individual's immune response.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

### REFERENCES: THE DISCLOSURES OF THE FOLLOWING REFERENCES ARE INCORPORATED BY REFERENCE

1. Chan MC, Kuok DI, Leung CY, et al. Human mesenchymal stromal cells reduce influenza A H5N1-associated acute lung injury in vitro and in vivo. Proc Natl Acad Sci U S A. 2016; 113(13):3621-3626. doi: 10.1073/pnas.1601911113
2. Loy H, Kuok DIT, Hui KPY, et al. Therapeutic Implications of Human Umbilical Cord Mesenchymal Stromal Cells in Attenuating Influenza A(H5N1) Virus-Associated Acute Lung Injury [published correction appears in J Infect Dis. 2019 Jan. 7; 219(2):339]. J Infect Dis. 2019; 219(2): 186-196. doi: 10.1093/infdis/jiy478

What is claimed is:

1. A high-throughput in vitro lung injury model system for screening a plurality of candidate compounds for the treatment of respiratory infectious diseases, comprising:
a plurality of transwell inserts in a 96-well system, wherein each transwell insert contains a monolayer of primary human alveolar epithelial cells, and the primary human alveolar epithelial cells are infected with one or more respiratory pathogens;
a device for applying a plurality of candidate compounds to infected alveolar epithelial cells; and
an apparatus for measuring alveolar fluid clearance and alveolar protein permeability in the infected alveolar epithelial cells.

2. The high-throughput in vitro lung injury model system of claim 1, wherein the respiratory infectious diseases comprise viral respiratory infections.

3. The high-throughput in vitro lung injury model system of claim 1, wherein the one or more respiratory pathogens comprise influenza virus, respiratory syncytial virus (RSV), coronavirus, middle east respiratory syndrome coronavirus (MERS-COV), human parainfluenza viruses (HPIVs), human metapneumovirus (hMPV), adenoviruses, rhinoviruses, and enteroviruses.

4. The high-throughput in vitro lung injury model system of claim 1, wherein the primary human alveolar epithelial cells are isolated from a non-malignant lung tissue.

5. The high-throughput in vitro lung injury model system of claim 1, wherein the primary human alveolar epithelial cells are seeded in the transwell inserts at a density of 1×10⁴ to 1×10⁵ cells per well.

6. The high-throughput in vitro lung injury model system of claim 1, wherein the plurality of candidate compounds comprise nano-sized extracellular vesicles derived from human mesenchymal stromal cells.

7. A method for high-throughput screening of a plurality of candidate compounds for the treatment of respiratory infectious diseases, the method comprising:
isolating primary human alveolar epithelial cells from a non-malignant lung tissue;
seeding and culturing the primary human alveolar epithelial cells in a plurality of transwell inserts in a 96-well transwell system, wherein each transwell insert contains a monolayer of the primary human alveolar epithelial cells;
infecting the primary human alveolar epithelial cells with one or more respiratory pathogens;
treating infected primary human alveolar epithelial cells with a plurality of candidate compounds; and
assessing effect of the plurality of candidate compounds on at least one physiological parameter comprising alveolar fluid clearance (AFC) and alveolar protein permeability (APP) to screening candidate compounds.

8. The method of claim 7, wherein the respiratory infectious diseases comprise viral respiratory infections.

9. The method of claim 7, wherein the one or more respiratory pathogens comprise influenza virus, respiratory syncytial virus (RSV), coronavirus, middle east respiratory syndrome coronavirus (MERS-COV), human parainfluenza viruses (HPIVs), human metapneumovirus (hMPV), adenoviruses, rhinoviruses, and enteroviruses.

10. The method of claim 7, wherein the primary human alveolar epithelial cells are isolated from a non-malignant lung tissue.

11. The method of claim 7, wherein the plurality of candidate compounds comprise nano-sized extracellular vesicles derived from human mesenchymal stromal cells.

12. The method of claim 7, wherein step of measuring impact of the therapeutic compound on at least one physiological parameter comprising measuring inflammatory cytokine expression.

13. The method of claim 7, wherein step of measuring impact of the therapeutic compound on at least one physiological parameter comprising monitoring activity of one or more ion transporters selected from the group consisting of epithelial sodium channels (ENaC), CFTR channels, and Na/K-ATPase pumps.

14. The method of claim 7, wherein the primary human alveolar epithelial cells are cultured in a liquid-liquid interface within the 96-well transwell system.

15. The method of claim 7, wherein the primary human alveolar epithelial cells are seeded at a density of 1×10⁴ to 1×10⁵ cells per well.

16. The method of claim 7, wherein the primary human alveolar epithelial cells are infected with one or more respiratory pathogens at a multiplicity of infection (MOI) ranging from 0.1 to 3.

17. The method of claim 7, wherein the plurality of candidate compounds are administered at a concentration ranging from 1×10⁷ to 1×10⁹ EV particles for each well.
